(19) United States
(12) Patent Application Publication
(10) Pub. No.: US 2006/0253047 A1
(43) Pub. Date: Nov. 9, 2006

(54) Device for non-invasively detecting or monitoring a medical condition in a mammal

(76) Inventor: Michael James Fox, Rutland (GB)

Correspondence Address: MCANDREWS HELD & MALLOY, LTD, 500 WEST MADISON STREET, SUITE 3400, CHICAGO, IL, 60661, US

(21) Appl. No.: 10/549,689

(22) PCT Filed: Mar. 16, 2004
(86) PCT No.: PCT/EP04/02703

§ 371 Date: Jul. 17, 2006

(30) Foreign Application Priority Data
Mar. 18, 2003 (GB) 0306127.2

(51) Int. Cl. A61B 5/00

(52) U.S. Cl. 600/584

(57) ABSTRACT

A device for non-invasively detecting or monitoring a medical condition in a mammal, said device comprising a member (2, 3, 10) adapted to be worn upon the body of the mammal to receive at least some of a bodily fluid excreted by the mammal, said member carrying one or more marker ingredients (4) which interact with one or more components of the bodily fluid to generate a colour or other visible indication, said interaction being characteristic of the medical condition in the mammal.

The present invention relates to a device for non-invasively detecting or monitoring a medical condition in a mammal.

### BACKGROUND TO THE INVENTION

In order to detect or monitor a medical condition in a human being or other mammal, it is often necessary to draw a sample of the blood of the mammal and to subject that to one or more tests to identify an illness or condition from which the mammal is suffering or to monitor the progress or that condition, for example its response to treatment. Many of such conditions also evidence themselves by way of the presence or absence of constituents in the urine or other bodily fluid excreted by the mammal so that collection of a sample of the bodily fluid and analysis or testing of that fluid can also be used to identify or monitor the condition. However, such methods are invasive in the case of taking blood samples or require collection of a sample of the excreted bodily fluid, which is cumbersome and often overlooked by the patient.

We have now devised a method for detecting or monitoring a medical condition in a mammal which reduces the above problems and provides simple and effective monitoring or detecting which is non-invasive and does not require conscious effort by the patient.

### SUMMARY OF THE INVENTION

Accordingly, the present invention provides a non-invasive method for detecting or monitoring a medical condition in a mammal, which method comprises detecting a visual and/or colour change in a marker ingredient which interacts with one or more components of a bodily fluid excreted by the mammal to generate a colour or other visible indication which interaction is characteristic of a medical condition in the mammal, the marker ingredient being carried by a carrier member worn by the mammal and which receives at least part of the bodily fluid excreted by the mammal.

The invention also provides a device for use in the method of the invention, which device comprises a member adapted to be worn upon the body of a mammal and to receive at least some of the bodily fluid to be assessed, the member carrying one or more marker ingredients which interact with one or more components of the bodily fluid to generate a colour or other visible indication which interaction is characteristic of a medical condition in the mammal.

The invention can be applied to detecting and/or monitoring a wide range of medical conditions in a range of mammals, for example kidney disorders in domestic animals, cattle or horses. However, the invention is of especial application in the monitoring and/or detection of medical conditions in humans, including the excretion of drug residues or metabolites and for convenience will hereinafter be described in terms of this preferred application.

The invention can be applied to the monitoring of an existing condition by detecting fluctuations in the interaction between the bodily fluid and a known level of marker ingredients on the carrier member. Alternatively, the invention can be applied to the routine screening of patients to detect an infection of malfunction of an organ. It is particularly preferred that the body fluid which is contacted with the marker ingredient be urine, but the fluid may be any other which can be conveniently collected by the carrier member and for which a marker ingredient gives a visible interaction which can be related to the existence or severity of a condition in the patient. For convenience, the invention will be described hereinafter in terms of testing the urine of a patient.

The invention is of especial benefit where there is patient resistance to the use of conventional non-invasive techniques. For example, the invention can be used with babies, infants, the aged or infirm where the collection of clean urine samples is difficult or where the patient readily overlooks or fails to carry out a routine testing programme. By collecting the urine and testing that, once the patient is persuaded to wear the carrier member, testing of the urine is inherent at each urination.

The invention can be applied to the detection of a wide range of components of the excreted bodily fluid which are indicators or medical conditions in humans. Thus, the invention can be applied to detecting sugars and sugar levels in the urine of a patient undergoing treatment for diabetes, to detecting urea, white blood cells or other indicators of infections and/or failure of the kidney or liver, cancer tumour markers, or blood in the urine. Marker ingredients for monitoring or detecting such and other conditions are commercially available and may be used in their commercially available forms in the present invention. The marker ingredients may be ones which interact directly with a component of the urine, for example in the detection of white blood cells or blood in the urine. Alternatively, the marker ingredient may be a combination of materials in which one ingredient interacts with a component of the urine to form an intermediate material which then interacts with another marker ingredient to generate the colour or visual change. An example of the latter is the interaction of glucose in the blood with glucose oxidase in the carrier member to form hydrogen peroxide which then reacts with tolidine in the carrier member to form a characteristic blue colour. For convenience, the invention will be described hereinafter in terms of the use of a mixture of glucose oxidase and tolidine to detect glucose in the urine.

The marker ingredient(s) are applied to the carrier medium in any suitable form so that collection of the urine from the patient brings the urine into contact with the marker ingredients. Thus, the carrier member may be a sheet of a suitable polymer, for example the other polyethylene sheet layer of a composite diaper of incontinence pad structure, having the marker ingredient(s) applied thereto in a suitable binder composition. Alternatively, the marker ingredient(s) may be absorbed into the body of the absorbent pad of such a diaper or incontinence pad. For example, the marker ingredient(s) may be applied to a component of the pad during construction of the pad using conventional powder deposition techniques as are used to apply particulate super absorber materials to enhance the fluid uptake of the pad. If desired, the marker ingredient(s) may be applied in separate stages so that the initial interaction of the glucose in the urine takes place at the outer layer or layers of the diaper and the resultant hydrogen peroxide then migrates into the body of the pad to interact with the tolidine within the body of the pad. However, for ease of detection of the colour or visual change in the marker ingredient(s), it is preferred to apply these as a continuous or discontinuous layer or coating to an outer or external component of the pad structure. It is particularly preferred to apply the active marker ingredients in a liquid carrier containing a resinous binder to the inner or outer face of the next-to-the-skin water permeable layer of the composite pad or diaper structure.

The diaper or pad typically will comprise a single or multi-layer pad of fibrilated cellulosic fibres sandwiched between an inner, next-to-the-skin, water permeable layer and an outer water impermeable layer. Such forms of construction are widely used in the manufacture of disposable diapers, sanitary pads or incontinence pads and it is particularly preferred to apply the marker ingredient(s) to the inner face of the water permeable sheet forming part of the next-to-the-skin layer of the diaper by roller or other applicators. It is particularly preferred to apply the marker ingredient(s) as a series of overlapping or discrete droplets using an ink jet printer.

As indicated above, the carrier member is preferably one of the layers of a composite absorbent pad diaper or incontinence pad which are to be worn by the patient, for example as a diaper, trainer pant or as an incontinence pad in a pair of water proof drawers or underpants. However, the invention can also be applied to other articles which are to be worn by the person, for example a colostomy or external urine collection bag. In such cases the bag is not in the form of an article of clothing as with a diaper, but is carried by the person and has the urine directly excreted into it. The term worn by the patient is therefore to be construed herein as including carrier members which are attached to the person and into or onto which the urine from the wearer is directly excreted and retained.

The marker ingredient(s) will usually be applied as a coating to part or all of that area of the carrier which is to receive the urine excreted from the patient. However, it is within the scope of the present invention for the carrier member to be a membrane or pad over or through which the urine flows to the main receptacle which retains the urine. For example, the marker ingredient(s) can be applied to the inner face of the tube feeding the urine to the colostomy bag or can be impregnated into a pad within such a tube or in the neck of the colostomy bag.

For convenience, the invention will be described hereinafter in terms of a coating of the marker ingredient(s) on the water permeable layer of the diaper or pad.

As indicated above, the marker ingredient(s) are preferably applied to the water permeable layer using a roller or other conventional fluid application technique. It is particularly preferred to apply the marker ingredient(s) in a liquid carrier, for example an aqueous or solvent carrier, using an ink jet printer, for example using the on-line technique described in our European Patent No 0211524B. The use of an ink jet printer technique allows the marker ingredient(s) to be deposited on the carrier member in any suitable pattern and at application rates which may vary across the surface of the carrier member.

The amount and type of marker ingredient(s) which are applied to the surface of the carrier member will depend upon the nature of the marker ingredient(s), the component of the urine which is being monitored or detected and may be readily determined by simple trial and error tests. If desired, the marker ingredient(s) may be deposited in a series of layers of different ingredients and the amount of marker ingredient(s) applied may vary over the surface of the carrier member to achieve different concentrations across the surface of the carrier medium.

In addition to the marker ingredient(s) the material applied to the carrier member may contain other ingredients to enhance the utility of the coating or layer applied to the carrier medium. For example, the material may contain one or more colour filter materials to screen out or reduce the effect of extraneous other ingredients. Thus, the material can incorporate a red colour filter medium so as to reduce the effect of blood in the urine during menstrual cycles on the blue colour generated when testing for glucose in the urine. The material applied to the carrier member may contain one or more slow or delayed release materials which progressively dissolve or break down so as to permit progressive access of the urine to the marker ingredient(s). In this way the blue colour for glucose in the urine can be generated by successive excretions of urine onto the carrier member and not just by the first excretion.

The invention thus provides a simple and effective means by which the presence of components in the urine of a patient can be monitored without the need for invasive blood sampling and which is inherently carried out at each urination by the patient without the need for the patient to remember to take any specific action.

### DESCRIPTION OF THE DRAWINGS

The invention will now be described by way of illustration with respect to a preferred embodiment thereof as shown in the accompanying drawings in which

FIG. 1 is a diagrammatic cross section through the absorbent pad of a disposable diaper or incontinence pad to be worn in the crotch area of a patient and to receive and contain urine excreted from the patient; and

FIG. 2 is a cross section through the mouth of a colostomy bag incorporating a pad of foamed plastic or other material impregnated with a marker ingredient and through which the urine must percolate on its travel from the patient to the interior of the colostomy bag.

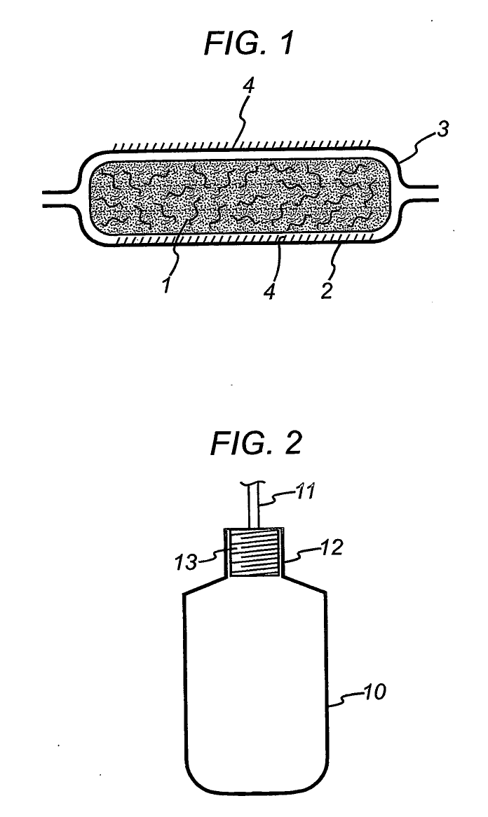

### DESCRIPTION OF THE PREFERRED EMBODIEMENT

The absorbent pad of a diaper or incontinence pad comprised a pad 1 of fibrilated cellulose fibres or other fibrous mass sandwiched between an outer water impervious polythene sheet layer 2 and a water pervious next-to-the-skin woven material layer 3. Many forms of such structure are known and used in the diaper or incontinence fields and are suitable for present use. A coating 4 of glucose oxidase and tolidine in a polyacrylic binder is applied to part or all of the layer 3 in the crotch region of the pad. The coating may be continuous or discontinuous and is preferably applied as an aqueous solution on line using an ink jet printer as the sheet of woven material 3 is fed to the process during which the composite fabric of the pad is formed during the manufacture of the diaper or incontinence pad. If desired, the coating can be dried using a hot air flow as the sheet 3 is fed into the diaper manufacture production process. Alternatively, the coating can be applied during production of sheet 3 prior to its subsequent transport to and use in the manufacture of the diaper.

In use, the pad is worn upon the crotch area of a patient and acts to receive and retain urine and faeces excreted by the patient. As the urine is excreted onto the layer 3 of the pad, any glucose in the urine reacts with the glucose oxidase in the coating 4 to release hydrogen peroxide which then reacts with the tolidine in the coating to generate a characteristic blue colouration. The patient or a nurse or other carer for the patient can then readily observe that this colour has been generated when the diaper or pad is removed from the patient. Where the coating contains the marker ingredient(s) incorporated into a slow release composition, this can be selected so that only some of the marker ingredient(s) are accessible initially by the urine and further marker ingredient(s) only become accessible at a later time. In this way, colour is developed initially in one area of the diaper or pad and subsequently in other areas enabling the user to determine the colour generation over a period of time and over more than one urine excretion.

If desired, the coating 4 can be formed on the inner face of the outer layer 2 in addition to or in replacement for the coating on layer 3 so that the generation of the blue colour can be observed through layer 2 without the need to remove the diaper or pad from the patient.

In the embodiment shown in FIG. 2, a patient wears a colostomy bag 10 connected by a tube 11 to his partially sectioned colon and excretes urine through tube 11 into bag 10. The neck 12 of the bag 10 is fitted with a foamed plastic or other porous or foraminous plug 13 which has been impregnated with the mixture of glucose oxidase and tolidine. As the patient urinates, the glucose in the urine passing through plug 13 causes the generation of the characteristic blue colour.

The generation of the blue colour in coating 4 or plug 13 alerts the patient or a nurse caring for the patient that he has glucose in his urine so that remedial action can be taken. Alternatively, where the patient is known to have glucose in his urine, for example due to diabetes, generation of a blue colour would be expected. However, a change in the hue of the colour indicates a change in the level of the glucose and hence a change in the patient's diabetic condition.

From another aspect, the invention thus provides a method for producing a carrier medium intended to be worn on a patient, which method comprises apply to that medium one or more marker ingredients which are to interact with a component in a bodily fluid which is excreted by the patient so as to generate a characteristic colour or other visible change.

The invention yet further provides a carrier medium to be worn upon the body of a mammal and having applied thereto one or more marker ingredients which are to interact with a component in a bodily fluid which is excreted by a mammal so as to generate a characteristic colour or other visible change.

1. A device for non-invasively detecting or monitoring a medical condition in a mammal, said device comprising a member adapted to be worn upon the body of the mammal to receive at least some of a bodily fluid excreted by the mammal, said member carrying one or more marker ingredients which interact with one or more components of the bodily fluid to generate a colour or other visible indication, said interaction being characteristic of the medical condition in the mammal.

2. A device according to claim 1 wherein said member comprises a bodily fluid absorbent pad sandwiched between an inner, next to the body, bodily fluid permeable layer, and an outer bodily fluid impermeable layer.

3. A device according to claim 2 wherein said one or more marker ingredients are applied to said inner layer.

4. A device according to claim 2 wherein said one or more marker ingredients are applied to the inner face of said outer layer, and are observable through said outer layer.

5. A device according to claim 2 wherein said one or more marker ingredients are applied to said absorbent pad.

6. A device according to claim 2 wherein said one or more marker ingredients comprises first and second marker ingredients, said first marker ingredient is applied to either or both of said inner and outer layers, and said second marker ingredient is applied to said absorbent pad, interaction of said bodily fluid with said first marker ingredient resulting in the migration into said absorbent pad of a substance which then interacts with said second marker ingredient to generate said colour or other visible indication.

7. A device according to claim 1 wherein said member comprises a bag or container worn by the mammal for collection of said bodily fluid.

8. A device according to claim 7 wherein said bag or container includes a component through which or over which said bodily fluid passes in receipt by said bag or container, said one or more marker ingredients being applied to said component.

9. A device according to claim 7 wherein a tube feeds said bodily fluid to said bag or container, and said one or more marker ingredients are applied to the inner face of the tube and/or a component within the tube.

10. A device according to any one of the preceding claims wherein said one or more marker ingredients are incorporated into a slow release composition so as to permit only progressive access of said bodily fluid to the marker ingredients thereby to provide detection or monitoring of said medical condition over a relatively lengthy period of time.

11. A device according to any one of the preceding claims wherein said member in addition to carrying said one or more marker ingredients also carries one or more colour filter materials to screen out or reduce the visual effect of extraneous components of said bodily fluid on said colour or other visual indication of said medical condition.

12. A device according to any one of the preceding claims wherein said member is adapted to be worn upon the body of a human to receive at least some of the urine excreted by the human.

13. A non-invasive method for detecting or monitoring a medical condition in a mammal, said method comprising detecting a visual and/or colour change in a marker ingredient which interacts with one or more components of a bodily fluid excreted by the mammal to generate a colour or other visible indication, said interaction being characteristic of the medical condition in the mammal, the marker ingredient being carried by a carrier member worn by the mammal which receives at least part of the bodily fluid excreted by the mammal.

14. A method for producing a device for non-invasively detecting or monitoring a medical condition in a mammal, which device comprises a member adapted to be worn upon the body of the mammal to receive at least some of a bodily fluid excreted by the mammal, said method comprising applying to that member one or more marker ingredients which are to interact with one or more components in the bodily fluid so as to generate a colour or other visible indication, said interaction being characteristic of the medical condition in the mammal.

15. A method according to claim 14 wherein said marker ingredients are applied to said member by means of ink jet printing and in a liquid carrier containing a resinous binder.
